SEQUENCE LISTING (1 sequence)

SEQ ID NO: 1 - protein, 5 aa, Artificial
  Chemically synthesized peptide fragment
  Xaa (1)..(5): Xaa is a non-natural amino acid
XFRWG

(19) United States
(12) Patent Application Publication
(10) Pub. No.: US 2007/0179279 A1
(43) Pub. Date: Aug. 2, 2007

(54) Methods for the synthesis of arginine-containing peptides

(76) Inventors: Lin Chen, Superior, CA (US); Christopher R. Roberts, Berthoud, CO (US)

Correspondence Address: Roche Colorado Corporation, 2075 North 55th Street, Boulder, CO, 80301, US

(21) Appl. No.: 11/646,779

(22) Filed: Dec. 28, 2006

Related U.S. Application Data
(60) Provisional application No. 60/755,132, filed on Dec. 30, 2005.

(51) Int. Cl. C07K 1/02

(52) U.S. Cl. 530/333

(57) ABSTRACT

Methods for the synthesis of arginine-containing peptides are provided. The methods include a step that minimizes contact of side chain protected arginine with base prior to the coupling step.

### CROSS-REFERENCE TO RELATED APPLICATIONS

The present non-provisional patent Application claims priority under 35 USC §119(e) from United States Provisional Patent Application having Ser. No. 60/755,132, filed on Dec. 30, 2005, and titled METHODS FOR THE SYNTHESIS OF ARGININE-CONTAINING PEPTIDES, wherein the entirety of said provisional patent application is incorporated herein by reference.

### FIELD OF THE INVENTION

The invention relates to the synthesis of peptides. More particularly, the invention relates to the synthesis of arginine-containing peptides.

### BACKGROUND OF THE INVENTION

Many methods for peptide synthesis are described in the literature (for examples, see U.S. Pat. No. 6,015,881; Mergler et al. (1988) Tetrahedron Letters 29:4005-4008; Mergler et al. (1988) Tetrahedron Letters 29:4009-4012; Kamber et al. (eds), Peptides, Chemistry and Biology, ESCOM, Leiden (1992) 525-526; Riniker et al. (1993) Tetrahedron Letters 49:9307-9320; Lloyd-Williams et al. (1993) Tetrahedron Letters 49:11065-11133; Andersson et al. (2000) Biopolymers 55:227-250; and Bray, B. L. (2003) Nature Reviews 2:587-593. The various methods of synthesis are distinguished by the physical state of the phase in which the synthesis takes place, namely liquid phase or solid phase.

Peptides and amino acids from which peptides are synthesized tend to have reactive side groups as well as reactive terminal ends. Undesired reactions at side groups or at the wrong terminal end of a reactant produces undesirable by-products, sometimes in significant quantities. These by-products and reactions can seriously impair yield or even ruin the product being synthesized from a practical perspective. To minimize side reactions, it is conventional practice to appropriately mask reactive side groups and (alpha amino) terminal ends of reactants to help ensure that the desired reaction occurs.

The use of amino acids having acid-cleavable alpha amino protecting groups such as Boc (N-Boc-AA representing a Boc alpha amino protected amino acid) have been used in peptide synthesis. In solid phase synthesis, N-Boc-AA can be coupled to the reactive end of a nascent peptide that is bound to a resin. After coupling, but prior to coupling the subsequent N-Boc-AA, the Boc protecting group is removed under acid conditions like TFA (trifluoroacetic acid). The group that links the peptide to the resin, and any side chain protecting group(s), should be resistant to TFA treatment. In Boc chemistry, side chain protecting group removal and peptide cleavage from the resin have traditionally been performed using a strong acid such as HF or TFMSA. Use of these types of strong acids can compromise the quality of the peptide product and can also be undesirable from a processing perspective. This is a significant disadvantage of Boc chemistry and is one reason for the present popularity of alternative chemistries such as Fmoc, which generally use gentler alpha amino cleavage conditions and offer a wide range of options for side chain protecting groups.

The use of Fmoc chemistry for protection of the alpha amino group has become the preferred method for most contemporary solid and solution phase peptide synthetic processes. Fmoc chemistry has also been shown to be more reliable and produce higher quality peptides than Boc chemistry. In Fmoc synthesis, removal of the Fmoc protecting group to provide a reactive amino terminus is typically performed in the presence of a mild base, such as piperidine. After base treatment, the nascent peptide is typically washed and then a mixture including an activated amino acid and coupling co-reagents is placed in contact with the nascent peptide to couple the next amino acid. After coupling, non-coupled reagents can be washed away and then the protecting group on the N-terminus of the nascent peptide can be removed, allowing additional amino acids or peptide material to be added to the nascent peptide in a similar fashion.

In Fmoc chemistry, reactive side chain groups of the amino acid and peptide reactants, including the resin-bound peptide material as well as the additional material to be added to the growing chain, typically remain masked with side chain protecting groups throughout synthesis. Generally, side chain protecting groups are used that are not removed during deprotection of the alpha amino protecting group (i.e., piperidine treatement) during synthesis. Commonly used side chain protecting groups in Fmoc chemistry are removable by acidolysis (e.g., using TFA) and include Acm, Boc, Mtr, OtBu, Pbf, Pmc, tBu, and Trt. In Fmoc chemistry, these protecting groups are available on certain amino acids, as permitted by the chemical structure of the side chain.

Despite the current widespread use of Fmoc chemistry in solid and solution phase processes, there are circumstances wherein using Fmoc chemistry is problematic. Such circumstances arise when the peptide requires the combination of certain amino acid residues. One problematic combination is found in peptides that are synthesized using both side chain protected arginine and tryptophan amino acids. It has been demonstrated that cleaved sulfonyl-based side chain protecting groups from arginine can react with the side chain of tryptophan. For example, by sulfonation of the tryptophan side chain by the by-products from the deprotection of Mtr-, Pmc-, and Pbf-protected arginine residues.

The extent of transfer of the cleaved arginine protecting group to the side chain of tryptophan depends on the spatial distance of these side chains. When these two amino acids are separated by one amino acid, the transfer of the cleaved arginine protecting group is the most pronounced, and it cannot be completely prevented by the use of currently utilized scavenger mixtures.

In attempts to overcome this problem it has been suggested that the protected amino acids Fmoc-Arg(Pmc)-OH or Fmoc-Arg(Pbf)-OH be used in conjunction with Fmoc-Trp(Boc)-OH.

Despite this, the use of side chain protecting groups such as Pmc and Pbf can present other problems. For example, extended deprotection conditions are often necessary to remove Pmc and Pbf following peptide synthesis. Extended deprotection conditions can be detrimental to peptide quality. As another example, slow reaction kinetics have been observed for coupling Fmoc-Arg (Pmc) amino acids (see, for example, U.S. Pat. No. 6,600,015). If slow reaction conditions lead to incomplete coupling, the coupling process may have to be repeated, or the synthesis of the peptide aborted. Alternatively, if synthesis of the peptide is continued, the peptide may be produced in very low yield. These situations are undesirable.

Furthermore, many amino acids with Fmoc alpha amino protecting groups and side chain protecting groups are expensive and, in some cases, require excess co-reagents to promote coupling. This can have a significant impact on the economic feasibility of peptide synthesis on a larger scale.

In order to avoid problems associated with the side chain protecting groups of arginine, side chain unprotected, alpha amino protected arginine is also available for use in peptide synthesis. However, as discussed, undesired reactions at side groups produces undesirable by-products. In the case of arginine, the tri-functional guanidino side chain is strongly nucleophilic and can be reactive in conditions wherein either side chain unprotected arginine is free in solution (not coupled to the nascent peptide) or coupled to the nascent peptide.

In the presence of a base (such as DIEA (diisopropylethylamine), which is commonly used in coupling reactions) arginine can self-cyclize as a result of nucleophilic attack by the deprotonated amine group of the unprotected side chain on the carbonyl group. This is undesirable as it prevents arginine from being coupled to the deprotected terminus of the nascent peptide.

Side chain unprotected arginine coupled to the nascent peptide is also problematic from the standpoint that an unprotected nitrogen on the side chain can be acylated during subsequent synthesis steps in the presence of an excess amount of base. This is also undesirable as peptide quality can be compromised.

Given this, and the current technologies known to those in the art, the synthesis of arginine-containing peptides presents a dilemma to the biochemist. The choice of either side chain protected arginine or side chain unprotected arginine can lead to problems that reduce the quality and/or the quantity of the peptide product.

The present invention addresses these problems and provides advances and improvements in the art of synthesizing arginine-containing peptides.

### SUMMARY OF THE INVENTION

The invention provides particularly effective and efficient methods for the preparation of arginine-containing peptides. The methods provide routes for overcoming difficulties in the synthesis of arginine-containing peptides by employing the use of side chain unprotected arginine in a coupling reaction in conjunction with one or more processing sub-steps that minimize self-cyclization of arginine. These methods allow for production of a high quality arginine-containing peptide while circumventing problems that would otherwise be associated with side chain protecting groups of arginine (for example, the reaction of a cleaved arginine side chain protecting group with the side chain of a tryptophan residue). The methods advantageously provide processing benefits associated with the use of side chain unprotected arginine.

In one aspect, a method of adding arginine to a nascent peptide is provided. The method comprises the steps of obtaining an inactive coupling composition comprising dissolved side chain unprotected arginine comprising an acid-removable alpha amino protecting group (e.g., BOC). Following this, the side chain unprotected arginine is utilized in a coupling reaction in the presence of an active coupling composition, wherein the side chain unprotected arginine is coupled to the amino terminus of a nascent peptide in the presence of base, such as DIEA. The base promotes the coupling reaction. The side chain unprotected arginine is either coupled to a nascent peptide that already includes a tryptophan residue, or the method further comprises a step of coupling a tryptophan residue to the nascent peptide after coupling of the side chain unprotected arginine. In preferred aspects, the nascent peptide is coupled to a resin.

The inactive coupling composition is obtained by avoiding contacting the side chain unprotected arginine with an amount of base conventionally used to promote a coupling reaction. In conventional approaches, contact with an amount of base (such as DIEA) useful for promoting amino acid coupling can lead to self-cyclization of the reactive arginine amino acid side chain as a result of nucleophilic attack by the deprotonated amine group on the carbonyl group. This can significantly reduce arginine coupling to the nascent peptide. This method minimizes the contact of the side chain unprotected arginine with the base prior to coupling of the side chain unprotected arginine to the nascent peptide, thereby improving the arginine coupling step. In turn, this improves the quality of the peptide product.

Minimizing contact of the side chain unprotected arginine with the base can be accomplished in one or more ways. In one preferred mode practice, the method includes the steps of preparing an inactive coupling composition including dissolved side chain unprotected arginine (optionally but preferably with co-reagents), adding the base to the inactive coupling composition to create an active coupling composition, and then contacting the nascent peptide with the active coupling composition without unnecessary delay.

The inactive coupling composition is prepared without an amount of base conventionally used for coupling, and the inactive coupling composition can include coupling co-reagents (such as HOBt and HBTU). The inactive coupling composition is preferably prepared at a low temperature. After dissolution of the side chain unprotected arginine and co-reagents, a base that promotes coupling of the side chain unprotected arginine to the nascent peptide, such as DIEA, is added to the inactive coupling composition to prepare the active coupling composition. The active coupling composition is then promptly placed in contact with the nascent peptide within a reasonable period of time, and arginine coupling is allowed to proceed.

In another mode of practice the nascent peptide is simultaneously contacted with the side chain unprotected arginine and the base, and arginine coupling is allowed to proceed.

In some aspects, the arginine-containing peptide is formed using a plurality of amino acids having acid-removable alpha amino protecting groups, wherein the amino acids other than the side chain unprotected arginine are also side chain unprotected. In these aspects, neither use of side chain protected amino acids nor global side chain deprotection is required, resulting in processing benefits.

In other aspects, the arginine-containing peptide is formed wherein one or more amino acids other than the side chain unprotected arginine are side chain protected. For example, the methods of the present invention can also provide a peptide comprising a side chain protected tryptophan residue and a side chain unprotected arginine residue. Advantageously, in one exemplary method, the side chain protecting group of the tryptophan residue is removed during the step of cleaving the peptide from the resin. For example, following coupling of the ultimate amino acid, the peptide is treated with an ammonia/methanol solution, causing removal of the tryptophan side chain protecting group and cleavage of the peptide from the resin. This method is also beneficial as harsh deprotection steps are not required.

In some aspects, the methods are utilized in a method for the synthesis of short arginine-containing peptides, such as those that are 10, 9, 8, 7, and 6 amino acids in length or less, and more preferably 5 amino acids in length or less. This is favorable when peptides are synthesized using side chain unprotected amino acids, as there is a reduced likelihood that undesirable reactions occur at the side chain. Short arginine-containing peptides synthesized using side chain unprotected amino acids can be produced according to the present methods in high quality.

In some aspects, the methods are utilized in a method for the synthesis of arginine-containing peptides, wherein a tryptophan residue is in sequential proximity to the arginine residue. For example, the arginine residue is immediately adjacent to a tryptophan residue, or the arginine residue and tryptophan residues are separated by one amino acid residue. These aspects represent an improvement over synthetic methods that provide a peptide wherein a tryptophan residue is in sequential proximity to the arginine residue, and the arginine residue includes a side chain protecting group.

Furthermore, the methods can also include one or more processing steps that minimize the contact of the nascent peptide with base following the step of coupling the side chain unprotected arginine. This also improves peptide quality by minimizing acylation of the unprotected arginine side chain. In one mode of practice, subsequent to arginine coupling, and between the step of alpha amino group (e.g., Boc) deprotection and amino acid coupling, the nascent peptide is not contacted with a base. As another measure for minimizing acylation, subsequent to arginine coupling, amino acid coupling steps do not use a molar excess of base.

One class of arginine-containing peptides that are particularly suitable for preparation by the methods of the invention are peptides that selectively stimulate melanocortin-4 (MC-4) receptor activity. MC-4 receptor agonist peptides are believed to be useful in treating or preventing obesity (Huzar, D., et al. (1997) Cell 88:131-41) and male erectile dysfunction (MED) (Sebhat, I. K., et al. (2002) J Med Chem. 45:4589-93). The synthesis of short arginine-containing pentapeptides that include a non-natural amino acid having high selectivity for the MC4 receptor have also been described in U.S. Pat. No. 6,600,015. These peptides are represented by the formula: AAₙₙ-(D)Phe-Arg-Trp-Gly-NH₂ (SEQ ID NO:1), wherein AAₙₙ represent a non-natural amino acid structure as described in the '015 patent. Preferred MC-4 agonist have a non-natural amino acid selected from 4-amino-1-phenylpiperidine-4-carboxylic acid and 4-amino-1-(2-methylphenyl)piperidine -4-carboxylic acid.

In some aspects, the invention provides a method of preparing a peptide having a sequence: AAₙₙ-(D)Phe-Arg-Trp-Gly-NH₂ (SEQ ID NO:1), wherein AAₙₙ represents a non-natural amino acid comprising a step of coupling a side chain unprotected arginine comprising an acid-removable alpha amino protecting group to a nascent peptide having the sequence Trp-Gly-R, wherein R represents resin. In one preferred mode of practice, the method includes the steps of preparing an inactive coupling composition including dissolved side chain unprotected arginine, adding the base to the inactive coupling composition to create a active coupling composition, and then promptly contacting Trp-Gly-R with the second composition.

The methods of the invention allow for the synthesis of arginine-containing peptides on any scale, including small and large (commercial) scale processes. In some modes of practice the methods are advantageously employed to provide improved commercial scale processes for the synthesis of arginine-containing peptides. The methods can provide such improvements as reduction in processing (synthesis) time, reduction in amount of reagents and starting materials required, as well as a reduction in the use of expensive reagents, and also improvements in peptide quality.

### DETAILED DESCRIPTION OF THE INVENTION

All publications and patents mentioned herein are hereby incorporated by reference in their respective entireties. The publications and patents disclosed herein are provided solely for their disclosure. Nothing herein is to be construed as an admission that the inventors are not entitled to antedate any publication and/or patent, including any publication and/or patent cited herein.

The embodiments of the present invention described below are not intended to be exhaustive or to limit the invention to the precise forms disclosed in the following detailed description. Rather, the embodiments are chosen and described so that others skilled in the art can appreciate and understand the principles and practices of the present invention.

The process of the present invention can be used to make peptides incorporating arginine and are especially useful at the stage when arginine is coupled to a nascent peptide. The term “nascent peptide” refers to a portion of a peptide that is in the process of being synthesized (i.e., subjected to amino acid coupling steps). The nascent peptide can be one amino acid residue, or more than one amino acid residue coupled via peptide bonds. The nascent peptide can be coupled to a resin (as prepared by solid phase synthesis) or non-resin bound (as prepared to solution phase synthesis).

More specifically, the methods described herein include a step of coupling a side chain unprotected arginine residue having an acid-removable alpha amino protecting group to a nascent peptide. Prior to the coupling step, the side chain unprotected arginine is subject to minimal contact with a base (i.e., the base that is used to promote the coupling reaction) before the arginine is coupled to the nascent peptide. This minimizes cyclization of arginine prior to and during the coupling step, and therefore improves the quality of the arginine containing peptide. Following coupling, and if arginine is not the last amino acid added to the nascent peptide, the acid-removable alpha amino protecting group can be removed from the coupled arginine, and a next amino acid can be coupled to the nascent peptide. One or more steps of coupling amino acids can be performed until a peptide having a desired sequence is formed.

The method of the invention can also be used to couple a side chain unprotected arginine to an activated resin. In this aspect, the methods can be used to provide a loaded resin and the nascent peptide.

The alpha amino protecting group is preferably TFA labile. In many modes of practice the alpha amino protecting group comprises an alkoxycarbonyl group, such as Boc. Boc-amino acids are commercially available in side chain unprotected and side chain protected forms.

The arginine-containing peptide can be synthesized as a full-length peptide (referring to peptides wherein no additional amino acids or peptide fragments are coupled to the peptide) or can be synthesized as an intermediate peptide. Intermediate peptides can be subject to one or more coupling steps with additional amino acids or peptide intermediate fragments to produce a peptide of greater length.

In some aspects, the arginine residue is coupled to the nascent peptide in a step that places the arginine residue near, or at, the terminal end of the peptide. For example, the arginine residue can be formed within 1-5 residues of the terminus of the peptide. For example, the arginine residue can be the final residue in the peptide sequence (ultimate), the second to last residue (penultimate), or the third to last residue (penpenultimate). The final three residues of the peptide may also include more than one arginine residue. The placement of a side chain unprotected arginine residue near, or at, the terminal end of the peptide can be beneficial from a peptide quality standpoint. Contact with base, which, in excess, may lead to acylation of a side chain nitrogen of the unprotected arginine residue, is minimized as the side chain unprotected arginine is subject to a limited number of steps that place base in contact with the nascent peptide having the side chain unprotected arginine residue.

In some aspects, the methods are used for the synthesis of a short arginine-containing peptide. For example, the methods can be employed for the synthesis of arginine-containing peptides that are 10, 9, 8, 7, or 6 amino acids in length or less, and more preferably 5 amino acids in length or less. Synthesis of shorter peptides may be particularly desired when peptides side chain unprotected amino acids are utilized in the coupling reaction, as there is a reduced likelihood that undesirable reactions occur on the unprotected side chains. In some aspects a peptide is synthesized wherein the coupling steps comprise a plurality of steps of coupling side chain unprotected amino acids.

While the methods of the present invention are particularly advantageous for the preparation of short peptides, the methods may also be included in a hybrid synthesis scheme. For example, a peptide intermediate fragment having a side chain unprotected arginine residue can be produced according to the present methods, which can be coupled to one or more other peptide intermediate fragments to provide a full length peptide product.

The amino acids from which the arginine-containing peptide can be derived can be naturally occurring amino acid residues, non-natural amino acid residues, or combinations thereof. The twenty common naturally-occurring amino acid residues are as follows: A (Ala, alanine), R (Arg, arginine); N (Asn, asparagine); D (Asp, aspartic acid); C (Cys, cysteine) Q (Gln, glutamine), E (Glu, glutamic acid); G (Gly, glycine); H (His, histidine); I (Ile, isoleucine); L (Leu, leucine); K (Lys, lysine); M (Met, methionine); F (Phe, phenylalanine); P (Pro, proline); S (Ser, serine); T (Thr, threonine); W (Trp, tryptophan); Y (Tyr, tyrosine); and V (Val, valine). Naturally occurring rare amino acids are also contemplated and include, for example, selenocysteine and pyrrolysine.

In some aspects, non-natural amino acids are included in the arginine-containing peptide. Non-natural amino acids include organic compounds having a similar structure and reactivity to that of naturally-occurring amino acids and include, for example, D-amino acids, beta amino acids, omega-amino acids (such as 3-aminopropionic acid, 2,3-diaminopropionic acid, 4-aminobutyric acid, and the like), gamma amino acids, cyclic amino acid analogs, propargylglycine derivatives, 2-amino-4-cyanobutyric acid derivatives, Weinreb amides of α-amino acids, and amino alcohols. In one aspect of the invention, and as described herein, a non-natural amino acid as described in U.S. Pat. No. 6,600,015 is used in the present methods in the synthesis of an arginine-containing peptide.

Residues of one or more other monomeric, oligomeric, and/or polymeric constituents optionally can be incorporated into the arginine-containing peptide. Non-peptide bonds may also be present. These non-peptide bonds can be between amino acid residues, between an amino acid and a non-amino acid residue, or between two non-amino acid residue. The alternative non-peptide bonds can be formed by utilizing reactions well known to those in the art, and may include, but are not limited to, imino, ester, hydrazide, semicarbazide, azo bonds, and the like.

The invention also contemplates methods of preparing arginine-containing peptides that have been chemically altered to contain one or more chemical groups other than amino acid residues, sometimes referred to as modified peptides. Such chemical groups can be included at the amino termini of the peptides, the carboxy termini, and/or at one or more amino acid residues along the length of the peptide. In still further embodiments, the peptide can include additional chemical groups present at their amino and/or carboxy termini, such that, for example, the stability, reactivity and/or solubility of the peptides are enhanced. For example, hydrophobic groups such as dansyl, acetyl, t-butyloxycarbonyl, or 9-fluorenylmethoxy-carbonyl groups can be added to the amino termini of peptides. Additionally, the hydrophobic group, t-butyl, or an amido group can be added to the carboxy termini of peptides. Similarly, a para-nitrobenzyl ester group can be placed at the carboxy termini of peptides. Techniques for introducing such modifications are well known in the art.

In a peptide synthesis process, a number of amino acid coupling steps are typically performed. In the present invention, one of the coupling steps includes coupling a side chain unprotected arginine having an acid removable alpha amino protecting group. In some cases, the side chain unprotected arginine can be coupled to a nascent peptide that has a tryptophan residue. In other cases, a tryptophan residue can be coupled to the nascent peptide after the step of coupling the side chain unprotected arginine.

In some aspects of the invention, the arginine-containing peptide is synthesized in a process wherein the amino acid coupling steps in the synthetic process include coupling side chain unprotected amino acids. In these cases, one or more steps of removing side chain protecting groups are not required. Therefore, some aspects of the invention provide methods for preparing arginine-containing peptides comprising a plurality of steps of coupling side chain unprotected amino acids. As indicated, if the process comprises a plurality of steps of coupling non-side chain protected amino acids, it is preferred that the peptide synthesized has a length of about 10, 9, 8, 7, or 6 residues or less, and more preferably 5 residues or less.

In some cases, and depending upon the type of reagents used in solid phase synthesis and other peptide processing steps, an amino acid may not require the presence of a side chain protecting group. This is typically the case when the side chain is non-reactive under standard synthesis conditions. Such amino acids typically do not include a reactive oxygen, nitrogen, or sulfur in the side chain.

Amino acids that do not include a reactive oxygen, nitrogen, or sulfur in the side chain are glycine, alanine, leucine, isoleucine, phenylalanine, and valine. In some aspects of the invention, it can be desirable to synthesize an arginine-containing peptide having one or more of these residues, as the chance of unwanted side chain reactions is further minimized. For example, in some embodiments of the invention, a peptide of 10, 9, 8, 7, or 6 residues or less, and more preferably 5 residues or less amino acid residues or less is prepared by a method that comprises the steps of (a) coupling a side chain unprotected arginine residue having an acid removable alpha amino protecting group, and (b) coupling a side chain unprotected residue selected from the group of glycine, alanine, leucine, isoleucine, phenylalanine, and valine having an acid removable alpha amino protecting group.

In other aspects of the invention, the method for synthesizing an arginine-containing peptide comprises one or more steps of coupling a side chain protected amino acid having an acid-removable alpha amino protecting group. In these aspects, the side chain protecting group is not removable under conditions that are used to remove the acid removable alpha amino protecting group. For example, the side chain protecting group should be compatible with alpha amino protected Boc amino acid chemistry.

A side-chain protecting group refers to a multi-atom chemical moiety covalently bonded to an atom of the side chain (R group in the general amino acid formula H₂N—C(R)(H)—COOH) of an amino acid. Side chain protecting groups do not include atoms ionically attached to an atom of the side chain. Side chain protecting groups have typically been used in synthetic methods to reduce the chance that a portion of the side chain will react with chemicals used in steps of peptide synthesis, processing, and the like.

Examples of Boc alpha amino protected amino acids having side chain protecting groups include Boc-Asp(OcHx)-OH, Boc-Cys(Acm)-OH, Boc-His(Dnp)-OH, Boc-Ser(Bzl)-OH, Boc-Trp(For)-OH, Boc-Asp(OBzl)-OH, Boc-Cys(4-MeBzl)-OH, Boc-Glu(OBzl)-OH, Boc-Thr(Bzl)-OH, Boc-Lys(2-Cl-Z)-OH, and Boc-Tyr(2-Br-Z)-OH.

In one mode of synthesis, the method includes a step of coupling a tryptophan that has a side chain protecting group, or the step of coupling the side chain unprotected arginine comprises coupling the arginine to a nascent peptide comprising a tryptophan residue. Commercially available side chain protected tryptophan amino acids include Boc-Trp(For)-OH. Removal of the formyl group (For) can be accomplished in the presence of a base (Merrifield et al. (1982) Biochemistry, 21:5020).

In one aspect, the method includes a step of coupling an amino acid having a side chain protecting group that is removable under conditions used for cleavage of the peptide from a resin in a solid phase synthesis approach.

In preferred modes of practice, the arginine-containing peptide is synthesized on a solid phase resin. The resin material can be formed from one or more polymers, copolymers, or combinations of polymers such as polyamide, polysulfamide, substituted polyethylenes, polyethylene glycol, phenolic resins, polysaccharides, or polystyrene. The resin can also be any solid that is sufficiently insoluble and inert to solvents, such as DCM (dichloromethane), DMF (dimethylformamide), NMP (N-methyl pyrrolidone), MeOH (methanol), that are used in peptide synthesis. The solid support typically includes a linking moiety to which the growing peptide is coupled during synthesis and which can be cleaved under desired conditions to release the peptide from the support. In preferred aspects the linker is cleaved using methanolic ammonia and provides a terminal amide group.

According to the methods of the present invention, suitable resins can include linking groups that are cleavable in the presence of a base. Examples of preferred resins on which the arginine-containing peptide can be synthesized include Merrifield resins, phenylacetamidomethyl (PAM) resins, and hydroxymethylbenzoic acid (HMBA) resins. Other contemplated resins include brominated PPOA resins and oxime resins. Commonly available resins are those that are used with Boc chemistry.

The starting resin can be an unloaded resin (not having a preloaded amino acid) or a pre-loaded resin, such as one that is pre-loaded with an amino acid residue that provides a desired C-terminal amino acid. Resins that are pre-loaded protected amino acids are commercially available and are generally preferred as starting materials. For example, pre-loaded resins attached to N-α-Boc protected amino acids can be obtained from Novabiochem/EMD Biosciences (San Diego, Calif). For example, the resin can be a Merrifield resin pre-loaded with a Boc-alpha amino protected amino acid. In some modes of practice, wherein an MC-4 peptide is synthesized, a preloaded resin having a N-α-Boc protected glycine can be obtained and used as starting material.

To further facilitate discussion of the invention, the term “resin,” in the context of the following discussion, generally refers to resin with coupled nascent peptide, unless otherwise noted. Therefore, a step of contacting a resin with a reagent is generally performed to affect the nascent peptide.

For solid phase synthesis, an appropriate reaction vessel can be chosen, depending on the desired quantity of arginine-containing peptide to be synthesized. Scaled up synthesis of peptide can be carried on in reaction vessels having features including filters, stirrers, temperature gauges, heating and/or cooling elements, reagent input and product export ports and conduits, inert gas inlet/bubbler mechanisms.

The reaction vessel can be pre-treated prior to addition of the resin in order to prevent reagents from non-specifically adhering to the interior walls of the vessel. For example, silanization reagents, such as dichlorodimethylsilane, can be added to the vessel along with a solvent, such as one that is compatible with the resin and that will be used during solid phase synthesis, such as DCM. After pre-treatment the vessel can be washed to remove residual silanization reagents.

In preparation for amino acid coupling, the resin can then be added to the reaction vessel and washed with a solvent, such as DCM, DCE (dichloroethane), chloroform, or the like. A commonly used solvent for Merrifield resins is DCM. DCM and similar solvents can cause resin swelling, thereby facilitating reaction conditions, such as amino acid coupling and protecting group cleavage.

Generally, based upon the amount of peptide product desired, a selected amount of resin can be added to the reaction vessel. Typically, the loading capacity of the resin is taken into consideration in determining the amount of peptide to be synthesized and the quantity of reagents used in peptide synthesis. Loading capacities for a Boc-protected amino acid-coupled Merrifield resins are typically in the range of about 0.2 mmol/g to about 1.6 mmol/g.

Prior to removal of the Boc protecting group, the resin can be swelled in a solvent, such as DCM. Swelling is generally achieved by using an excess of solvent. In one mode of practice, 1 parts resin to 5 parts solvent (as measured by weight), or greater, or more preferably 1 parts resin to 10 parts solvent, can be used to swell the resin. The resin can also be agitated and/or mixed to improve swelling. Resin swelling can be conducted at suitable temperature, such as in the range of about 18° C. to about 28° C. A period of an hour or less is generally sufficient for resin swelling. One resin pre-wash may be sufficient to swell the resin prior to adding a deprotection reagent; however additional pre-washes can be conducted if desired. The solvent can then be drained and then an acid can be added to the resin to cause removal of the Boc protecting group. Alternatively, an acid can be added to the solvent to start removal of the Boc group.

Boc group removal can be performed in the presence of a suitable acid, such as TFA. The acid can be used neat (undiluted) form, which may generally require shorter reaction times, or can be diluted with a suitable solvent, such as DCM. For example, ratios of 1:4 (acid:solvent) to 99:1 may be used. Boc removal can be conducted at a suitable temperature, such as in the range of about 18° C. to about 28° C. One or more than one acid treatments can be performed by contacting the nascent peptide bound to the resin with the acid, reacting for a period of time, removing the acid, and then repeating the cycle. Another option for alpha amino group removal is by contacting the nascent peptide bound to the resin with HCl in dioxane.

In one mode of practice, a mixture is prepared by diluting TFA in a solvent, such as methylene chloride, at a ratio of about 1:1. The resin is left in contact with the acid and agitated for a short period of time, such as about 20 minutes or less. The acid is then removed and the step of acid treatment is repeated. In one mode of practice the resin is treated multiple times with acid/solvent mixtures, wherein each subsequent treatment is longer than the previous one, preferably each treatment shorter than 20 minutes.

The acid treatment can be carried out to remove preferably most, or all of the alpha amino terminal protecting groups.

Following removal of the Boc protecting group the resin can be washed in solvent to remove residual acid. In one mode of practice the resin is washed with a solvent such as DCM multiple times using short incubations. The resin can also be washed with a solvent that is similar to, or the same as a solvent that is used in the subsequent coupling step. For example, the resin can be washed with DMF prior to the coupling steps.

Optionally, and preferably prior to addition of the side chain unprotected arginine, the resin may be contacted with a base between (a) the step of contacting the resin with an acid to remove the alpha amino protecting group from the nascent peptide, and (b) the step of coupling the amino acid to the nascent peptide chain. The base can neutralize acid remaining in contact with the resin. This can minimize end capping of the resin.

Exemplary bases include those used for promoting coupling, such as DIEA. In one mode of practice, prior to coupling an arginine residue to the nascent peptide chain, the resin is contacted with a mixture of a base and a solvent, such as a mixture of DIEA and DMF, to neutralize any acid from a previous step of alpha amino protecting group removal. For example, the base can be present in the solvent in an amount of 10%. Washes following base contact can be performed to remove any acid or base in unneutralized or neutralized form.

Following acid treatment for alpha amino protecting group removal and a desired wash process (with optional base treatment), the nascent peptide chain having unprotected terminal amino acid groups is contacted with an amino acid comprising an alpha amino protected group in a coupling step.

In describing amino acid coupling, the method of preparing the arginine containing peptide can include one step, or more than one step, of coupling an amino acid to the nascent peptide, prior to the step of coupling the side chain unprotected arginine. Any suitable solid or solution phase coupling reaction, including those described herein, can be performed to provide a nascent peptide prior to the step of coupling the side chain unprotected arginine.

In some aspects, the method includes a step of coupling tryptophan to the nascent peptide prior to the step of coupling the side chain unprotected arginine. For example, the tryptophan can be coupled to the nascent peptide in a coupling step that immediately precedes the arginine coupling step.

In some modes of practice, the method includes a step of coupling a side chain unprotected tryptophan. For example, Boc-Trp is coupled to the nascent peptide in a step preceding coupling the side chain unprotected arginine.

In other modes of practice the method includes a step of coupling a side chain protected tryptophan. For example, Boc-Trp(For) is coupled to the nascent peptide in a step preceding coupling the side chain unprotected arginine.

According to the invention, the method of adding arginine to a nascent peptide comprises a step of obtaining an inactive coupling composition comprising side chain unprotected arginine comprising an acid-removable alpha amino protecting group The method also comprises a step of coupling the side chain unprotected arginine to a nascent peptide in the presence of an active composition.

According to the invention, measures are taken to minimize base contact with the side chain unprotected arginine leading into the step of coupling the side chain unprotected arginine to the nascent peptide. In other words, in preparing for the arginine coupling reaction, the amount of time that the base is in contact with the side chain unprotected arginine prior to arginine being coupled to the resin, is minimized. In some aspects, minimizing contact can effectively be achieved by delaying contacting the side chain unprotected arginine with the base. Minimizing base contact improves the arginine coupling step as cyclization of the arginine side chain is significantly reduced.

The sequence of steps minimizes contact of the arginine residue with the base (e.g., DIEA), which activates coupling when the arginine is in contact with the resin.

The step of obtaining a composition can be performed by first preparing an inactive coupling composition. The inactive coupling composition can includes (a) side chain unprotected arginine having an acid labile alpha amino protecting group and optionally (b) one or more co-reagents, in a suitable solvent that does not promote arginine self-cyclization. For example, the first composition can be prepared by combining co-reagents such as phosphonium and uronium salts and auxiliary nucleophiles with side chain unprotected arginine in a suitable solvent, such as DMF.

The one or more co-reagents can be compounds that enhance or improve the coupling reaction. Compounds that increase the rate of reaction and reduce the rate of side reactions include phosphonium and uronium salts. In the presence of a base, for example, diisopropylethylamine (DIEA) or triethylamine (TEA), these co-reagents convert protected amino acids into activated species (for example, BOP, PyBOPO, HBTU, and TBTU all generate HOBt esters). Other co-reagents help prevent racemization by providing a protecting reagent. These reagents include carbodiimides (for example, DCC or WSCDI) and added auxiliary nucleophiles (for example, 1-hydroxy-benzotriazole (HOBt), 1-hydroxy-azabenzotriazole (HOAt), or HOSu).

Generally, the alpha amino acid protected, side chain unprotected arginine and co-reagents are used in an amount in molar excess of the amount of amino acid/nascent peptide coupled to the resin. For example, in relation to the amount of amino acid/nascent peptide coupled to the resin, more than one molar equivalent of the side chain unprotected arginine, and one or more co-reagent can be used. In one mode of practice about two molar equivalents of side chain unprotected arginine and one or more co-reagents are used.

In one mode of practice the inactive coupling composition includes the alpha amino acid protected, side chain unprotected arginine, HOBt, and HBTU. In one mode of practice the inactive coupling composition has a weight ratio of solids (e.g., amino acid and co-reagents) to solvent of about 1:5 (i.e., 1 g solids:5 g solvent) or greater, such as 1:10.

The inactive coupling composition is prepared in a manner to provide dissolved side chain unprotected arginine in the composition. This can be accomplished by allowing the side chain unprotected arginine (and any optional co-reagent) to mix with the solvent so that at least most of the side chain unprotected arginine is dissolved in the solvent.

The inactive coupling composition is generally prepared without an amount of base that is conventionally used to promote coupling of amino acid to the terminus of the nascent peptide during the coupling step. In some preferred modes of practice the inactive coupling composition either has “no base” or “substantially no base.” It is understood that in some instances small amounts of base may be present in the first composition, such as in the form of an impurity. If a composition has “substantially no base,” any base present is in an amount that does not unduly affect the quality of the side chain unprotected arginine (such as promoting by self-cyclization).

Prior to addition of the base, and in one preferred mode of practice, the temperature of the inactive coupling composition is preferably reduced to a temperature in the range of −5° C. to 5° C. This lower temperature can reduce cyclization of the arginine side chain after the base has been added but prior to coupling of the arginine to the nascent peptide by slowing the rate of reaction.

As stated, the method of the invention also comprises a step of coupling the side chain unprotected arginine to a nascent peptide in the presence of an active coupling composition. In one mode of practice, the base is added to the composition that includes the side chain unprotected arginine.

For example, after the side chain unprotected arginine and co-reagents are dissolved, the base can be added to the inactive coupling composition to create the active coupling composition. Preferably the base is a non-ionic nitrogen base such as DIEA, DBN (1,5 diazabicyclo[4.3.0]non-5-ene), or DBU (1,8 diazabicyclo[5.4.0]undec-7-ene). In one preferred aspect, the base is added in a molar amount that is not greater than the molar amount of the side chain unprotected arginine. In the presence of the base the co-reagents convert the side chain unprotected arginine in

The active coupling composition containing the base is generally added to the resin promptly after addition of the base. For smaller scale processes the time between base addition and resin contact may be less than a minute; however, for larger scale processes, the time between base addition and resin contact may be somewhat longer, for example, within five minutes, such as about three to five minutes, in order to adequately mix the base to provide the active coupling composition.

This sequence of steps minimizes, and delays, the amount of time that the base is in contact with the arginine, and therefore improves peptide quality by minimizing arginine self-cyclization. Side chain unprotected arginine is first dissolved, and then base is introduced. Prior to being coupled to the nascent peptide, some self-cyclization of the side chain unprotected arginine may occur; however, based on the methods of the invention, the amount of any self-cyclization does not significantly impact the quality of the peptide.

This mode of practice is illustrated, and compared to a conventional approach for amino acid coupling, in FIG. 1. “Boc-Arg” represents side chain unprotected, alpha amino Boc protected arginine, “Co-R” represents coupling co-reagents, “B” represents base as used for activation of the coupling reaction, “R” represents resin with bound nascent peptide, and the symbol “→” denotes a period of time, but does not necessarily represent a specific period of time.

[image]

Other approaches to minimizing contact of the side chain unprotected arginine and the base can also be employed. For example, a first (inactive) coupling composition containing side chain unprotected arginine and co-reagents, and a second composition that includes a base used to promote the coupling reaction, are prepared. The first and second composition can then be simultaneously added to a resin. When combination of the first and second composition becomes the active coupling composition.

In the coupling step, the active coupling composition comprising alpha amino acid protected, side chain unprotected arginine is typically combined with the resin for a period of time sufficient for the majority of the arginine to be coupled to the resin. For example, in some modes of practice, the amino acid can be place in contact with the nascent peptide chain for a period of about 2 to 3 hours at a temperature in the range of about 18° C. to about 28° C. Agitation can be performed during the coupling step.

The reaction can be monitored to determine the extent of coupling, for example, by Kaiser test.

Following the step of coupling the side chain unprotected arginine, one or more steps of coupling an amino acid can be performed. These reaction conditions can also be used for coupling other amino acids to the nascent peptide. However, it is noted that for amino acids other than the side chain unprotected arginine, it is not generally not necessary to minimize contact of the uncoupled amino acid with the base prior to contacting the resin.

However, measures can be taken to limit base contact with the unprotected side chain of the coupled arginine residue on the nascent peptide subsequent to the coupling step. This also improves peptide quality by minimizing acylation of the unprotected arginine side chain. For example, subsequent to arginine coupling, and between the step of alpha amino group (e.g., Boc) deprotection and amino acid coupling, the nascent peptide is not contacted with a base. As another measure for minimizing acylation, subsequent to arginine coupling, amino acid coupling steps do not use a molar excess of base.

After coupling has been determined to be complete, the spent coupling composition can be removed from the reaction vessel, for example, by draining the solvent. The resin with nascent peptide chain having the recently coupled amino acid can then be washed with one or more solvents, preferably multiple times. After coupling the arginine residue it is preferred that the resin be washed thoroughly to remove as much base as possible.

Washings can be conducted relatively quickly, such as less than 15 minutes for each wash. In one mode of practice the resin is washed multiple times with the solvent used in the coupling reaction, such as DMF, and then multiple times with a solvent that is used in the step of alpha amino protecting group removal, such as DCM.

This series of steps provides a nascent peptide comprising a side chain unprotected arginine. In some cases, wherein tryptophan is coupled to nascent peptide prior to coupling the side chain unprotected arginine, the nascent peptide comprises a side chain protected arginine residue and a tryptophan residue (either side chain unprotected or side chain protected).

After the resin has been washed, the resin can be subject to treatment with an acid to remove the alpha amino protecting group, as described herein.

One or more coupling steps with a subsequent amino can then be performed. The amino acid can be another side chain unprotected arginine (in which it is recommended to use the series of steps to minimize base contact prior to amino acid coupling) or can be an amino acid that is different than the arginine. If tryptophan is not coupled prior to coupling the side chain unprotected arginine, it can be coupled to the nascent peptide in one or more coupling steps subsequent to the arginine coupling. As indicated, the coupling conditions can be the same as those used for coupling the arginine residue.

In some aspects, a non-natural amino acid can be included in a coupling step. In preparing MC-4 peptides, the non-natural amino acid can represent the final amino acid to be coupled to the nascent peptide chain.

After the final amino acid has been coupled to the nascent peptide, it can be cleaved from the resin.

In some aspects, cleavage results in a peptide product having a C-terminal amide group. For example, the amide group can be generated by treating the a nascent peptide attached to the resin of the following formula: [peptide]-[C(O)O]-[R] with a base in a solvent, such as an ammonia/methanol solution to provide a peptide with a C-terminal acetamide group: peptide-[C(O)-NH₂].

In aspects wherein a residue on the peptide contains a side chain protecting group, the cleavage step preferably results in removal of the side chain protecting group.

Following cleavage, peptide purification can be performed. Any suitable purification method can be performed, including chromatography purification, such as HPLC. 20 While the inventive methods can be used for the synthesis of any sort of peptide having an arginine residue, the synthesis of the MC-4 peptide as described in described in U.S. Pat. No. 6,600,015, which exemplifies a short arginine-containing peptide, is discussed. Arginine-containing peptides that can be synthesized according to the present invention include those that are represented by formula I: 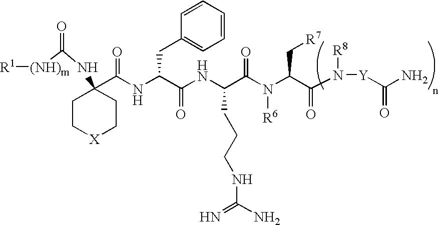
wherein m is 0 or 1, n is 0 or 1, R¹ is an unsubstituted linear or branched alkyl having from 1 to 8 carbon atoms; linear or branched alkyl having from 1 to 8 carbon atoms mono-substituted by phenyl or carboxyl; unsubstituted phenyl; or phenyl mono-substituted by fluoro, chloro or linear or branched alkyl having from 1 to 4 carbon atoms.
X is:

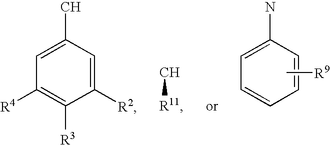

R^{2,} R³ and R⁴ are independently hydrogen or a linear or branched alkoxy having from 1 to 4 carbon atoms, wherein when R³ is alkoxy, R² and R⁴ are both hydrogen. R⁹ is hydrogen, linear or branched alkyl having from 1 to 3 carbons, linear or branched alkoxy having from 1 to 3 carbons, or unsubstituted phenoxy. R¹¹ is cyclohexyl, cycloheptyl, or a branched alkyl having from 3 to 8 carbon atoms. R⁶ is hydrogen or methyl. R⁷ is 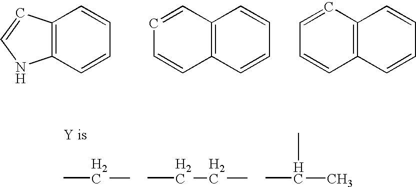
and R⁸ is hydrogen or methyl; or
Y is 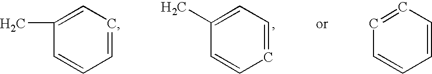
and R⁸ is hydrogen.

The methods of the invention can also be used to synthesize a compound of formula II:

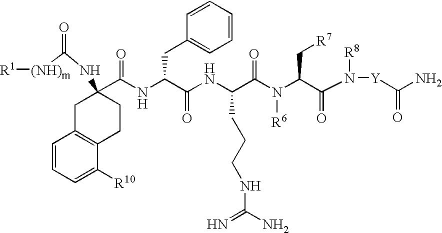

In the compounds of formula II, m is 0 or 1, n is 0 or 1, R¹ is an unsubstituted linear or branched alkyl having from 4 to 8 carbon atoms; linear or branched alkyl having from 1 to 8 carbon atoms mono-substituted by phenyl or carboxyl; or unsubstituted phenyl; or phenyl mono-substituted by fluoro, chloro or linear or branched alkyl having from 1 to 4 carbon atoms. R⁷ is 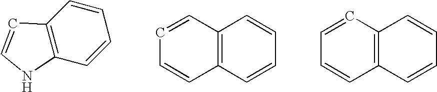 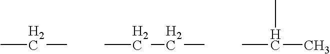
and R⁸ is hydrogen or methyl; or
Y is 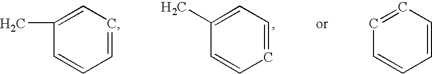
and R⁸ is hydrogen.

R¹⁰ is hydrogen, halo, linear or branched alkyl having from 1 to 3 carbon atoms, linear or branched alkoxy having from 1 to 3 carbon atoms, or —NR¹² N¹³ wherein R¹² and R¹³ are each independently a linear or branched alkyl having from 1 to 3 carbons or together are —(CH2)_{q}— wherein q is 3, 4 or 5.

For purposes of describing methods of the present invention, synthesis of a compound of formula AAₙₙ-(D)Phe-Arg-Trp-Gly-NH₂ (SEQ ID NO:1) is described, wherein AAₙₙ represent a non-natural amino acid as described in the '015 patent. Exemplary non-natural amino acids are 4-amino- 1-phenylpiperidine-4-carboxylic acid: 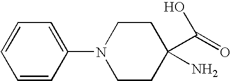
and derivatives thereof including 4-amino-1-(2-methylphenyl)piperidine-4-carboxylic acid:

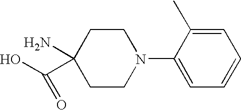

It has been discovered that by using alpha amino Boc protected, side chain unprotected arginine, a significant overall improvement in synthesis of peptides of the formula AAₙₙ-(D)Phe-Arg-Trp-Gly-NH₂ (SEQ ID NO:1) can be achieved. The side chain unprotected arginine can be used in combination with the specified sequence of base (e.g., DIEA) addition during the coupling step.

In one mode of practice, the method comprises a series of steps of coupling amino acids having acid-removable alpha amino protecting groups to provide a resin [R]-coupled sequence of Phe-Arg-Trp-Gly-[R].

In this series of steps, a step of coupling Trp to Gly-[R] comprises coupling either a side chain unprotected, or a side chain protected Boc-tryptophan to an alpha amino deprotected Gly-[R].

The series of steps also includes a step of coupling Arg to Trp-Gly-[R], which comprises preparing a first composition including Boc alpha amino protected, side chain unprotected arginine and coupling co-reagents, but without base. After the reagents are dissolved and the first composition is cooled, a base is added to prepare a second composition. The second composition is then promptly added to the alpha amino deprotected Trp-Gly-[R]. Next, a side chain unprotected Phe amino acid is coupled to Arg-Trp-Gly-[R] to provide Phe-Arg-Trp-Gly-[R]. The final coupling step includes coupling a non-natural amino acid (AAₙₙ) of the formula: 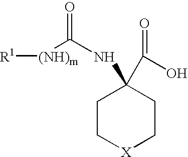
wherein R¹, m, and X are as defined herein, to deprotected Phe-Arg-Trp-Gly-[R], and then cleaving the AAₙₙ,-(D)Phe-Arg-Trp-Gly from the resin with a base to provide the formula AAₙₙn-(D)Phe-Arg-Trp-Gly-NH₂ (SEQ ID NO:1).

### EXAMPLE 1

### Solid Phase Synthesis of MC-4 Pentapeptide Using Boc Chemistry and Resin Cleavage

Solid phase synthesis to generate Me-APPC-Phe-Arg-Trp-Gly-Resin was performed. All reagents were obtained from Advanced Chemtech (Louisville, Ky) unless otherwise noted.

A 500 mL peptide synthesizer with integral bottom frit filter equipped with a mechanical stirrer, thermometer, and nitrogen inlet/bubbler was pre-treated with a mixture of 400 mL DCM and 0.8 mL dichlorodimethylsilane. The solution was stirred for 30 min, drained and washed two times with 400 mL of DCM.

To the reactor was added 10 g of Boc-Gly-Merrifield-resin (loading 1.1 mmol/g (Boc-Gly/resin)). The resin was then swelled in 10× volume (100 mL) of DCM for 30-60 min at 23° C., and then the DCM was drained.

A tryptophan residue was coupled to the Boc-Gly-Merrifield-resin as follows. Boc removal was accomplished by three treatments of 10× solution (100 mL) of 50%TFA/DCM(v/v) at 23° C. The treatments lasted 5, 15, 15 min. respectively. The 50%TFA/DCM solution was drained after each treatment. The resin was then washed with DCM three times (10 × volume (100 mL), 5 min, each at 23° C.), followed by three DMF washes (10× volume (100 mL), 5 min, each at 23° C.). To prepare the coupling solution of the amino acid, 2 equiv. of Boc-Trp (6.7 g), HOBt (2.94 g), and HBTU (8.34 g) were weighed out and dissolved in 10× volume (100 mL) of 10% DIEA/DMF(v/v) for 15 min at 5° C. The resultant solution was then mixed with the resin under agitation for 2-3 hours at 23° C. The sample was pulled for Kaiser test to check the reaction completion. After the coupling reaction was complete, the coupling solution was drained. The resin was washed with both DMF and DCM four times each (10× volume (100 mL), 5 min, each at 23° C.).

An arginine residue was coupled to the Boc-Trp-Gly-Merrifield-resin as follows. Boc removal was accomplished by three treatments of 10× solution (100 mL) of 50%TFA/DCM (v/v). The treatment lasted 5, 15, 15 min respectively at 23° C. The 50%TFA/DCM solution was drained after each treatment. The resin was then washed by DCM three times (10× volume (100 mL), 5 min, each at 23° C.), followed by two DMF washes (10× volume (100 mL), 5 min, each at 23° C.), a 10% DIEA/DMF (v/v) wash (10× volume (100 mL), 5 min, each at 23° C.), and DMF(v/v) wash (10× volume (100 mL), 5 min, each at 23° C.). To prepare the coupling solution 2 equiv. of Boc-Arg(HCl)-OH (6.84 g), HOBt (2.97 g), and HBTU (8.34 g) were weighed out and dissolved in 10× volume (100 mL) of DMF at 5° C. DIEA (at 2 equiv) (3.14 g) was later added in. The resultant solution was then promptly mixed with the resin under agitation for 2-3 hours at 23° C. A sample was pulled for Kaiser test to check the reaction completion. After the coupling reaction was completed, the coupling solution was drained and the resin was washed with both DCM and DMF four times each (10× volume (100 mL), 5 min, each at 23° C.).

A phenylalanine residue was coupled to the Boc-Arg-Trp-Gly-Merrifield-resin as follows. Boc removal was accomplished by three treatments of 10× solution (100 mL) of 50%TFA/DCM (v/v). The treatment lasted 5, 15, 15 min respectively at 23° C. The 50%TFA/DCM solution was drained after each treatment. The resin was then washed by DCM three times (10× volume (100 mL), 5 min, each at 23° C.), followed by three DMF washes (10× volume (100 mL), 5 min, each at 23° C.). To prepare the coupling solution of the amino acid, 2 equiv. of Boc-D-Phe (5.84 g), HOBt (2.97 g), and HBTU (8.34 g) were weighed out and dissolved in 10× volume (100 mL) of 10% DIEA/DMF(v/v) for 15 min at 5° C. The resultant solution was then mixed with the resin under agitation for 2-3 hours at 23° C. A sample was pulled for Kaiser test to check the reaction completion. After the coupling was complete, the coupling solution was drained. The resin was washed with both DMF and DCM four times each (10× volume (100 mL), 5 min, each at 23° C.).

The non-natural amino acid residue Me-APPC-OH (SEAC Chimie Fine, Gennevilliers, France) was coupled to the Boc-Arg-Trp-Gly-Merrifield-resin as follows.

Boc removal was accomplished by three treatments of 10× volume (100 mL) of 50%TFA/DCM(v/v) at 23° C. The treatment lasted 5, 15, 15 min respectively. The 50%TFA/DCM solution was drained after each treatment. The resin was then washed by DCM three times (10× volume, 5 min, each at 23° C.), followed by three DMF washes (10× volume (100 mL), 5 min, each at 23° C.). To prepare the coupling solution of the amino acid, 1.2 equiv. of Me-APPC-OH, HOBt, and HBTU were weighed out and dissolved in 10× volume (100 mL) of 10% DIEA/DMF(v/v) for 15 min. The resultant solution was then mixed with the resin under agitation for 16-24 hours at 23° C. The sample was pulled for Kaiser test to check the reaction completion. After the coupling reaction was judged complete, the coupling solution was drained. The resin was washed with both DMF and DCM four times each (10× volume (100 mL), 5 min, each at 23° C.).

The built MC-4 resin was washed with MeOH for four times (10× volume, 5 min, each). The last MeOH wash was not drained immediately. The MeOH suspension of the resin was cooled to a temperature in the range of −5° C. to −10° C. The MeOH was drained and the pre-prepared cold (at least at −10° C.) NH₃/MeOH solution (10 x volume, 1:2, v/v) was stirred with the resin for at least 1 hour. Then the reaction mixture was allowed to warm to 23° C. with continued agitation for another 2 h. The cleavage solution was collected and the resin was washed with MeOH for four times (10× volume, 5 min, each). The combined MeOH washes were combined with cleavage solution and distilled at reduced pressure. The resultant crude MC-4 was obtained as a yellow solid.

What is claimed is:

1. A method of adding arginine to a nascent peptide comprising steps of:
obtaining an inactive coupling composition comprising dissolved side chain unprotected arginine comprising an acid-removable alpha amino protecting group, and
coupling the side chain unprotected arginine to a nascent peptide in the presence of an active coupling composition, wherein the nascent peptide comprises a tryptophan residue, or the method further comprises a step of coupling a tryptophan residue to the nascent peptide comprising the side chain unprotected arginine.

2. The method of claim 1 wherein the step of obtaining, the inactive coupling composition comprises one or more coupling co-reagents.

3. The method of claim 1 further comprising a step of preparing the active coupling composition comprising the sub-step of adding base to the inactive coupling composition.

4. The method of claim 3 wherein the step of obtaining the inactive coupling composition comprises cooling the inactive coupling composition to a temperature in the range of −5° C. to 5° C. prior to the step of preparing the active coupling composition.

5. The method of claim 2 wherein the step of obtaining the inactive coupling composition comprises the co-reagents HOBt and HBTU.

6. The method of claim 1 wherein the step of coupling comprises contacting the nascent peptide chain with the active coupling composition.

7. The method of claim 1 wherein the step of coupling comprises coupling the side chain unprotected arginine to a nascent peptide that is linked to a resin.

8. The method of claim 6 wherein the step of cleaving comprises cleaving the nascent peptide from the resin with a base using a mixture of ammonia and methanol.

9. The method of claim 1 wherein the tryptophan residue on the nascent peptide is side chain unprotected, or the step of coupling the tryptophan residue to the nascent peptide comprises coupling a side chain unprotected tryptophan.

10. The method of claim 1 wherein the tryptophan residue on the nascent peptide is side chain protected, or the step of coupling the tryptophan residue to the nascent peptide comprises coupling a side chain protected tryptophan.

11. The method of claim 1 comprising a step of cleaving the acid-removable alpha amino acid protecting groups with TFA.

12. The method of claim 1 wherein the step of coupling the arginine residue comprises coupling BOC-arginine HCl.

13. The method of claim 1 where, in the step of obtaining, the inactive coupling composition comprises DMF.

14. The method of claim 1 wherein the step of coupling, the active coupling composition comprises a molar amount of the base that is not greater than a molar amount of the side chain unprotected arginine.

15. The method of claim 1, wherein prior to the step of coupling, the method further comprises a step of providing a nascent peptide comprising an unprotected alpha amino terminus wherein there is substantially no base in contact with the nascent peptide.

16. The method of claim 9 further comprising a step of coupling a side chain unprotected amino acid that is different than the side chain unprotected arginine and side chain unprotected tryptophan.

17. The method of claim 16 further comprising steps of coupling side chain unprotected amino acids to provide a peptide having no side chain protecting groups.

18. The method of claim 1 comprising steps of coupling amino acids to provide a peptide of 10 amino acids or less.

19. The method of claim 18 comprising steps of coupling amino acids to provide a peptide of 5 amino acids or less.

20. The method of claim 1 wherein the arginine residue is the penpenultimate residue.

21. The method of claim 1 comprising a step of coupling a non-natural amino acid subsequent to the step of coupling the side chain unprotected arginine residue.

22. The method of claim 1 comprising coupling steps to provide a peptide having a sequence: AAₙₙ-(D)Phe-Arg-Trp-NH₂, wherein AAₙₙ represents a non-natural amino acid.

23. The method of claim 22 wherein the non-natural amino acid comprises the formula: 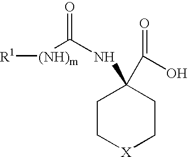
wherein m is 0 or 1, n is 0 or 1, R¹ is an unsubstituted linear or branched alkyl having from 1 to 8 carbon atoms; linear or branched alkyl having from 1 to 8 carbon atoms mono-substituted by phenyl or carboxyl; unsubstituted phenyl; or phenyl mono-substituted by fluoro, chloro or linear or branched alkyl having from 1 to 4 carbon atoms.
X is: 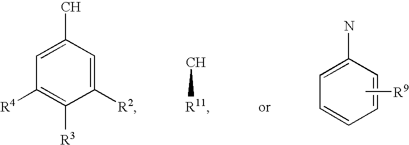
R², R³ and R⁴ are independently hydrogen or a linear or branched alkoxy having from 1 to 4 carbon atoms, wherein when R³ is alkoxy, R² and R⁴ are both hydrogen. R⁹ is hydrogen, linear or branched alkyl having from 1 to 3 carbons, linear or branched alkoxy having from 1 to 3 carbons, or unsubstituted phenoxy. R¹¹ is cyclohexyl, cycloheptyl, or a branched alkyl having from 3 to 8 carbon atoms. R⁶ is hydrogen or methyl. R⁷ is 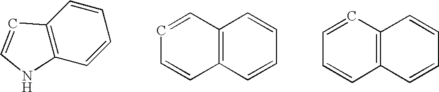
Y is 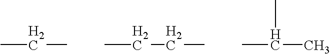
and R⁸ is hydrogen or methyl; or
Y is 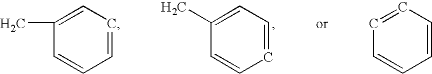
and R⁸ is hydrogen.

24. The method of claim 23 wherein the non-natural amino acid is selected from 4-Amino-1-phenylpiperidine-4-carboxylic acid and 4-amino-1-(2-methylphenyl)piperidine -4-carboxylic acid.

25. A method of preparing an arginine—and tryptophan-containing peptide comprising steps of:
coupling tryptophan comprising an acid-removable alpha amino protecting group to a nascent peptide to provide a tryptophan-containing nascent peptide,
preparing an inactive coupling composition comprising dissolved side chain unprotected arginine comprising an acid-removable alpha amino protecting group, and
coupling the side chain unprotected arginine to the tryptophan-containing nascent peptide comprising a tryptophan residue in the presence of an active coupling composition.

26. A method of preparing a peptide having a sequence: AAₙₙ-(D)Phe-Arg-Trp-Gly-NH₂ (SEQ ID NO:1), wherein AAₙₙ represents a non-natural amino acid comprising a step of coupling a side chain unprotected arginine comprising an acid-removable alpha amino protecting group to a nascent peptide having the sequence Trp-Gly-R, wherein R represents resin.
